(19) United States
(12) Patent Application Publication
(10) Pub. No.: US 2014/0154182 A1
(43) Pub. Date: Jun. 5, 2014

(54) N,N-SUBSTITUTED GUANIDINE COMPOUND

(71) Applicants: Pieter Jacob Klein, Amsterdam (NL); Athansios Metaxas, Amsterdam (NL); Albert Dirk Windhorst, Amsterdam (NL); Johannes Antonius Maria Christiaans, Amsterdam (NL); Bart Nicolaas Maria van Berckel, Amsterdam (NL)

(72) Inventors: Pieter Jacob Klein, Amsterdam (NL); Athansios Metaxas, Amsterdam (NL); Albert Dirk Windhorst, Amsterdam (NL); Johannes Antonius Maria Christiaans, Amsterdam (NL); Bart Nicolaas Maria van Berckel, Amsterdam (NL)

(73) Assignee: STICHTING VU-VUMC, Amsterdam (NL)

(21) Appl. No.: 14/122,691

(22) PCT Filed: May 30, 2012
(86) PCT No.: PCT/NL2012/050377

§ 371 Date: Jan. 29, 2014

(30) Foreign Application Priority Data
May 31, 2011 (NL) 2006875

(51) Int. Cl. C07C 279/18; C07C 277/08; A61K 51/04

(52) U.S. Cl. CPC C07C 279/18; A61K 51/04; C07C 277/08
USPC 424/1.89; 564/238; 424/1.81; 564/239; 424/1.85

(57) ABSTRACT

The invention is directed to a N,N-substituted guanidine compound or a salt or solvate thereof according to formula (1), R¹RNC(NH)NR²R³, wherein R¹ is methyl and R² is hydrogen. R³ is a organic group comprising a halogen and thiomethyl substituted phenyl group. R is an organic group comprising a substituted aryl group Z wherein the substituent group is —Y—R⁴, wherein Y is a heteroatom chosen from the group consisting of O, S and N and R⁴ is a fluorinated organic group.

The invention is directed to a N,N-substituted guanidine compound, to its manufacture and use as a medicament and as part of a radiopharmaceutical formulation.

WO-A-95/20950 describes a wide range of possible N,N-substituted guanidine compounds which according to this publication may be useful as a pharmaceutical active compound for treating a disorder of the nervous system in which the pathophysiology of the disorder involves excessive or inappropriate release of a neurotransmitter from neuronal cells. The labelled compounds may also be useful for diagnosing a selected disease wherein the pathophysiology involves ion-channel excitation or activity.

U.S. Pat. No. 5,637,622 and U.S. Pat. No. 6,251,948 also describe a range of possible N,N-substituted guanidine compounds which exert neuroprotective activity. The neuroprotective activity is achieved in that the compounds act as blockers for the ion channel of the N-methyl-D-aspartate (NMDA) receptor. Compounds with a high affinity to the ion channel pore of the NMDAR complex are preferred.

Journal of Labelled Compounds and Radiopharmaceuticals 2002, 955-964 describes the synthesis of [¹¹C]N-(2-chloro-5-thiomethylphenyl-N′-(3-methoxyphenyl)-N′-methylguanidine, also referred to as [¹¹C]GMOM. This article showed that this compound has a good affinity to the ion channel pore of the NMDAR complex.

Bioorganic Medicinal Chemistry Letters 2010, 1749-1751 describes N-(2-chloro-5-(S-fluoromethyl)thiophenyl)-N′-(3-thiomethylphenyl)-N′-methylguanidine and N-(2-chloro-5-(S-2-fluoroethyl)thiophenyl)-N′-(3-thiomethylphenyl)-N′-methylguanidine with a similar structure as GMOM but with an even better affinity to the ion channel pore of the NMDAR complex.

US2010/0143252 describes radiolabelled N-(2-chloro-5-methylthio)-phenyl-N′-(3-[18F]fluoromethylthio)-phenyl-N′-methylguanidine and their use for imaging an NMDA-mediated disease.

U.S. Pat. No. 6,153,604 describes N-(2,5-dibromo)-phenyl-N′-(3-trifluoromethoxy)-phenyl-N′-methylguanidine and N-(2-bromo 5-ethyl)-phenyl-N′-(3-trifluoromethoxy)-phenyl-N′-methylguanidine as compounds having an affinity to the ion channel pore of the NMDAR complex as expressed by its Ki value.

The object of the present invention is to provide a N-substituted guanidine compound having a good affinity to the ion channel pore of the NMDAR complex.

This object is achieved by the following compound. A N,N-substituted guanidine compound or a salt or solvate thereof according to formula (I)

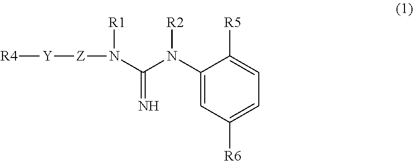

wherein R⁴ is a fluorinated organic group, Y is O, S or N,
Z is a substituted aryl group,
R¹ is methyl,
R² is hydrogen,

### R⁵ is Cl or Br and

R⁶ is a thiomethyl group.

Applicants found that these compounds according to the above have an improved affinity to the ion channel pore of the NMDAR complex. The compounds further have a low affinity to the sigma receptors which is advantageous because selectivity of binding is an important prerequisite of radiopharmaceuticals and it has been described previously for this class of compounds that besides binding to the NMDA receptor, binding to sigma sites occurs. These compounds act as non-competitive blockers for the ion channel of the NMDAR complex. The invention shall be described below describing preferred embodiments and further advantages of the present invention.

The fluorinated organic group R⁴ in formula (I) preferably has 1 to 5 carbon atoms and more preferably one carbon atom. The heteroatom Y in formula (I) is chosen from the group consisting of O, S and N, preferably chosen from the group consisting of O and S, and even more preferably Y is O. The number of fluor atoms present in group R⁴ is preferably 1 to 3, more preferably 3 and even more preferably 2. R⁴—Y— is preferably a mono, bi or tri-fluorinated methoxy group.

Aryl group Z may be further substituted. Preferably aryl group Z is not further substituted. Z is preferably a phenyl or naphthyl group and more preferably a phenyl group. The phenyl group is suitably substituted with the R⁴—Y— group at is 3-position.

R⁵ is Cl or Br and preferably Cl.

A preferred compound is when R⁴—Y— is a mono, bi or tri-fluorinated methoxy group, Z is a phenyl group substituted at its 3-position with the R⁴—Y— group and R⁵ is Cl. More preferably R⁴—Y is a bi-fluorinated methoxy group or a tri-fluorinated methoxy group.

The invention is also directed to the salts and solvates of the compounds described above. Suitable salts according to the invention, include physiologically acceptable acid addition salts such as those derived from mineral acids, but not limited to, hydrochloric, hydrobromic, phosphoric, metaphosphoric, nitric or sulphuric acids or those derived from organic acids such as, but not limited to, tartaric, fumaric, malonic, citric, benzoic, trifluoroacetic, lactic, glycolic, gluconic, methanesulphonic or p-toluenesulphonic acids.

The compounds according to the invention may advantageously be used as part of a pharmaceutical composition for use in the therapeutic treatment of neuronal loss in hypoxia, hypoglycemia, brain or spinal cord ischemia, and brain or spinal chord trauma as well as being useful for the treatment of epilepsy, Alzheimer's disease, Amyotrophic Lateral Sclerosis, Parkinson's disease, Huntington's disease, Down's Syndrome, Korsakoff's disease and other neurodegenerative disorders.

The radiolabelled compounds according to the invention can advantageously be used as diagnostic imaging agents for in vivo imaging of the ion channel of the NMDAR complex with positron emission tomography (PET) or single photon emission computed tomography (SPECT). The invention is thus directed to the use of said compound as an ion channel blocker of the N-methyl-D-aspartate (NMDA) receptor. The invention is thus also directed to these radiolabelled compounds, wherein at least one of groups, R¹, R², R⁴, R⁵, R⁶ of the guanidine group contains a radio-isotope selected from ³H, ¹¹C, ¹⁸F, ⁷⁶Br, ¹²³I, ¹²⁴I, ¹²⁵I, ¹³¹I, ¹¹C or ¹⁸F labelling are preferred. ¹¹C labelling is suitably applied to the methyl carbon of group R¹ of the guanidine moiety or to the alkyl carbon in the thio methyl group of group R⁶. ¹⁸F labelling suitably is applied to one fluor atom of group R⁴. Most preferred is a compound wherein one fluor atom in group R⁴ is the radio-isotope ¹⁸F or wherein the carbon of methyl group R¹ is the radio-isotope ¹¹C.

The NMDAR complex belongs to the ionotropic glutamate receptor family and are involved in many physiological processes. NMDAR's are heteromeric complexes which consists of four subunits namely three subtypes, NR¹, in eight different splice variants, NR², in four different subunits, NR³, in two different subunits (NR¹, NR² and NR³ are typical codes used in literature for describing NMDAR's and are not to be confused with R¹ and R² as used in formula (1)). Imaging the NMDAR complex in living animal or human brain by PET or SPECT provides useful information on the role of the NMDAR complex in various neurological disorders such as Alzheimer's disease, Huntington's disease, Korsakoff's disease and other neurodegenerative disorders, for example those described above.

The invention is thus also directed to a method for the in vivo diagnosis or imaging of NMDA related disease in a subject, preferably a human, comprising administration of a radiolabelled compound according to the invention. Administration of the compound is preferably administrated in a radiopharmaceutical formulation comprising the compound or its salt or solvate and one or more pharmaceutically acceptable excipients in a form suitable for administration to humans. The radiopharmaceutical formulation is preferably an aqueous solution additionally comprising a pharmaceutically acceptable buffer, a pharmaceutically acceptable solubiliser such as, but not limited to, ethanol, tween or phospholipids, pharmaceutically acceptable stabilizer solutions and/or antioxidants such as, but not limited to, ascorbic acid, gentisic acid or p-aminobenzoic acid.

The invention is thus also directed to a radiopharmaceutical formulation comprising the radiolabelled compound according to the invention and to a radiopharmaceutical formulation comprising the radiolabelled compound according to the invention for use as an in vivo diagnostic or imaging method, wherein the method is preferably positron emission tomography (PET) or single photon emission computed tomography (SPECT).

The compounds according to formula (I) may be prepared according to procedures described N. L. Reddy et al., Journal of Medicinal Chemistry (1994), 37, 260-267 and schematically shown below, wherein R¹ is an alkyl group, preferably methyl.

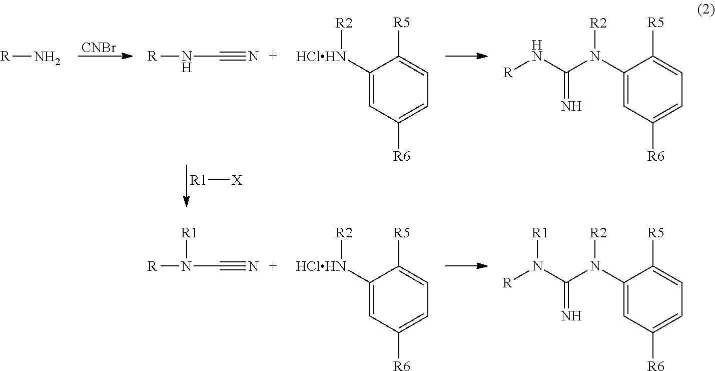

The radiolabelled compounds have a relatively short half time and are thus preferably prepared shortly before use in the above referred to in vivo diagnosis or imaging of NMDA related diseases. Suitably the compound is synthesised for the greater part to obtain a non-radiolabelled precursor compound. This non-radiolabelled precursor compound can by means of a relatively simple synthesis be reacted with a radiolabelled compound to obtain the radiolabelled compound of the present invention. The invention is also directed to any novel precursor described below.

Precursor compounds for preparing compounds according to formula (I) are suitably compounds wherein the ¹¹C comprising group R¹ in formula (I) is replaced by hydrogen and wherein the hydrogen group R² in formula (I) is replaced by an amine protecting group (P). Thus the suited precursor has hydrogen substituted for R¹ in formula (I) and an amine protecting group substituted for group R² in formula (I) in order to obtain the above described compounds wherein R¹ is methyl and R² is hydrogen. Suitable amine protecting groups are t-butyloxy carbamate, Fmoc, pivaloyloxymethyl, carboxybenzyl or any other selected from “Greene's Protective Groups in Organic Synthesis, by P. G. M Wuts and T. W. Greene”; and the other is hydrogen.

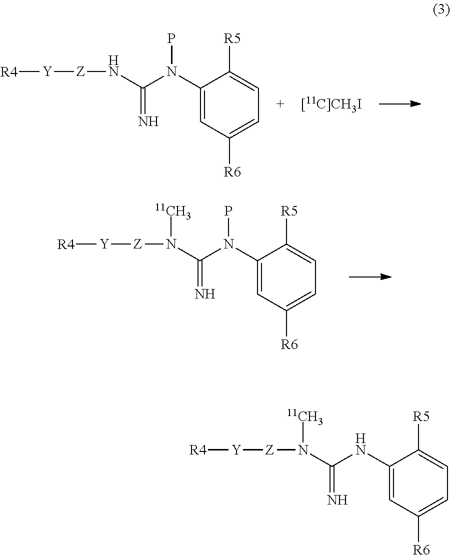

Precursor compounds for preparing a compound having a [¹¹C]— or [¹⁸F]— labelled R⁴ or R⁶ substituent in formula (I) respectively are preferably precursor compounds according to formula (I) wherein the hydrogen group R² is substituted by an amine protecting group (P).

Applicants found that the ¹¹C or ¹⁸F radiolabelled compound can also be made without using a protecting group. The invention is therefore also directed to the novel compounds 1-(2-chloro-5-(methylthio)phenyl)-3-(3-(difluoromethoxy)phenyl)guanidine and 1-(2-chloro-5-(methylthio)phenyl)-3-(3-(trifluoromethoxy)phenyl)guanidine and to their use as intermediated to prepare a ¹¹C labeled 1-(2-chloro-5-(methylthio)phenyl)-3-¹¹C-methyl-3-(3-(difluoromethoxy)phenyl)guanidine or 1-(2-chloro-5-(methylthio)phenyl)-3-¹¹C-methyl-3-(3-(trifluoromethoxy)phenyl)guanidine respectively.

The group R⁴—Y— or R⁶ which is to comprise the radio labelled atom is suitably substituted by a hydroxyl, thiol or amine group. The other group R⁴—Y— or R⁶ is a group described above for R⁴—Y— or R⁶ respectively. Below reaction equation (4) illustrates the synthesis for preparing a compound wherein R⁴ is [¹⁸F]— labelled and wherein Y in the below equation is O, S or N and L is a leaving group such as alkyl or aryl sulfonate, like, but not limited to, mesylate, triflate, tosylate or nosylate or halogen like bromine, iodine or chlorine.

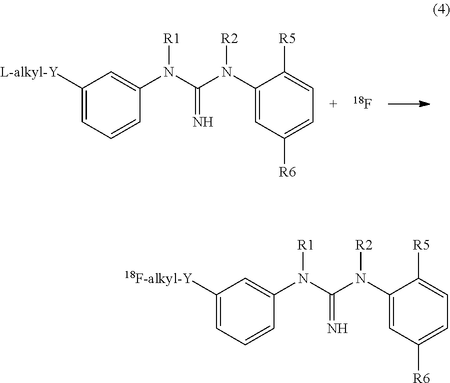

Below reaction equation (5) illustrates the synthesis for preparing a compound wherein R⁶ is [¹⁸F]— labelled and wherein Yin the below equation is O, S or N and L is a leaving such as alkyl or aryl sulfonate, like, but not limited to, mesylate, triflate, tosylate or nosylate or halogen like bromine, iodine or chlorine.

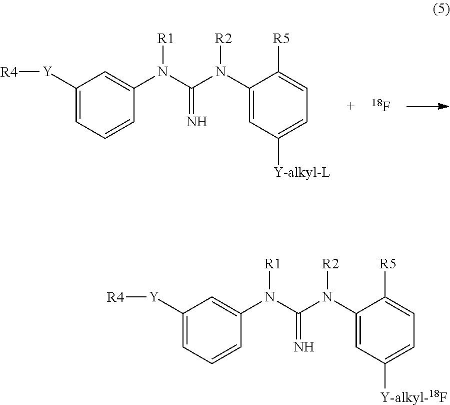

The precursor compound is preferably subjected to a nucleophilic fluorination, preferably carried out by heating or microwave irradiation of said precursor compound with [¹⁸F]fluoride complexed with a phase transfer catalyst such as (nBu)₄NHCO₃ or 4,7,13,16,21,24-Hexaoxa-1,10-diazabicyclo[8.8.8]hexacosane (Kryptofix[2.2.2]) in combination with or without a suitable base such as, but not limited to, potassium carbonate, potassium hydrogen carbonate, cesium carbonate in a suitable solvent such as, but not limited to, acetonitrile, N,N-dimethylformamide (DMF), dimethylsulfoxide (DMSO), sulfolane, ethanol, t-butanol or ionic liquids.

The deprotection reaction is preferably carried out in the presence of a suitable acid such as, but not limited to, hydrochloric acid, hydrogen bromide, trifluoro acetic acid or sulphuric acid; or a suitable base such as, but not limited to, sodium acetate, potassium hydroxide or sodium hydroxide or by a hydrogenation process in presence or in absence of a suitable catalyst such as, but not limited to, catalyst based on platinum, palladium, rhodium, ruthenium and nickel.

The radiolabelled compound according to the invention can be prepared by alkylation (6) of the above precursor compounds with substituted or unsubstituted, straight or branched [¹⁸F]fluoroalkyl-L, wherein L is selected from halogen, preferably chloro, bromine or iodo or another suitable leaving group such as alkyl or aryl sulfonate, like, but not limited to, mesylate, triflate, tosylate or nosylate.

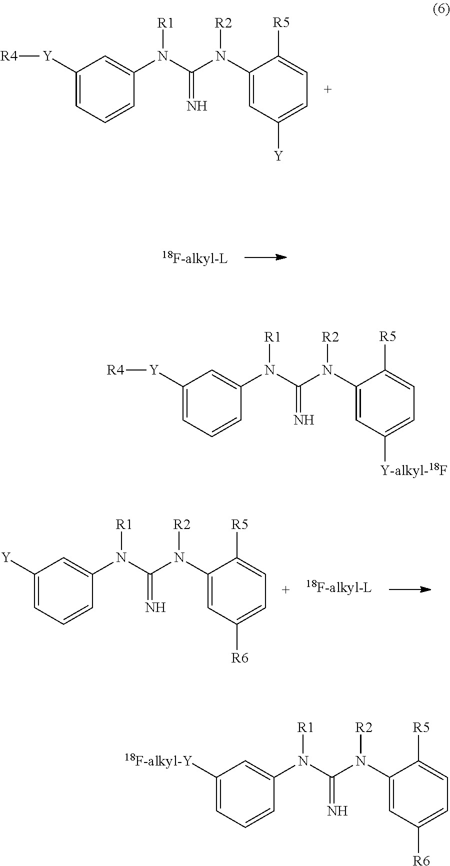

The alkylation reaction with the appropriate alkylhalide is preferable carried out in a suitable solvent such as, but not limited to, acetone, acetonitrile, t-butanol, chloroform, dichloromethane, N,N-dimethylformamide (DMF), dimethylsulfoxide (DMSO), ethanol, isopropanol, methanol, propanol or tetrahydrofuran (THF) and in presence of a suitable base such as, but not limited to, cesium carbonate, potassium carbonate, potassium hydrogen carbonate, potassium hydroxide or sodium hydride, t-butylammonium hydroxide, triethylamine, diisopropylamine, diisopropylethylamine or dimethylaminopyridine and in presence or absence of a suitable catalyst such as, but not limited to, sodium iodide or potassium iodide.

The deprotection reaction is preferably carried out as described above.

The invention is thus also directed to a process for the preparation of a ¹⁸F radio labelled N,N-substituted guanidine compound by
(i) reaction of a precursor in a suitable solvent and in the presence of a base with an [¹⁸F]fluoroalkyl-L, wherein L is a leaving group; wherein the precursor is a compound according to formula (I) wherein the hydrogen group R² is optionally exchanged by an amine protecting group and wherein on the R⁴—Y position a hydroxyl group is present on the aryl group Z, and
(ii) removal of the protection amine group in a suitable solvent and protective group removing reagent provided that R² is exchanged by an amine protecting group.

Preferably the precursor is 3-(2-chloro-5-(methylthio)phenyl)-1-(3-hydroxyphenyl)-1-methylguanidine.

The invention is also directed to the use of 3-(2-chloro-5-(methylthio)phenyl)-1-(3-hydroxyphenyl)-1-methylguanidine to prepare a ¹⁸F radio labelled N,N-substituted guanidine compound according to the present invention.

Compounds (7) having a guanidine moiety comprising a [¹¹C]-labelled atom is preferably prepared by reaction of the appropriate amine with [¹¹C]cyanic bromide ([¹¹C]CNBr), yielding a [¹¹C] labelled intermediate which is sub sequentially reacted with the appropriate amine hydrogen-halogen salt according to one of the two equations below:

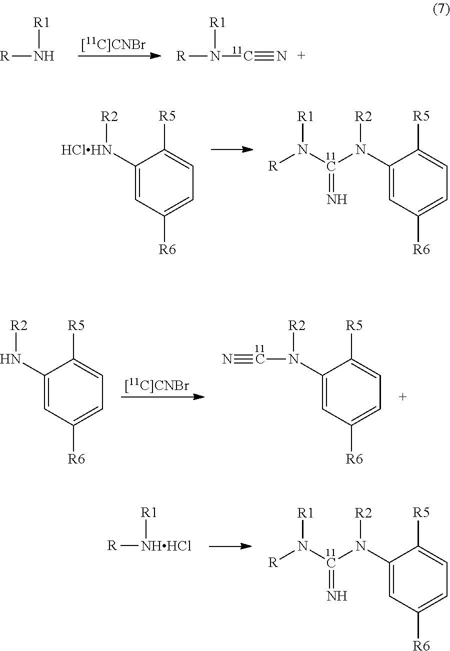

The reaction of the appropriate amine with [¹¹C]CNBr is preferably carried out at room temperature, by heating or microwave irradiation in combination or without a suitable base such as, but not limited to, NaHCO₃, CH₃COONa, KHCO₃, KHCO₃, triethylamine or, di-isopropylethylamine in a suitable solvent such as, but not limited to, diethylether, ethanol, THF, acetic acid, water, dichloromethane, toluene, chlorobenzene, N,N-dimethyl formamide, dimethyl sulfoxide or sulfolane. The [¹¹C] labelled intermediate and the appropriate amine hydrogen-halogen salt are reacted in a suitable solvent, preferably a high boiling solvent, such as, but not limited to, toluene, chlorobenzene, N,N-dimethyl formamide, dimethyl sulfoxide or sulfolane by heating or microwave irradiation.

The radio labelled compounds and non-radio labelled compounds according to the present invention may be purified according to those methods known to the person skilled in the art, for example by means of HPLC purification or Solid Phase Extraction (SPE). The HPLC purification is preferable carried out on a preparative HPLC column packed with reverse phase material such as, but not limited to, C18, C18-EPS or C8, a mobile phase consisting of a mixture of methanol, ethanol or acetonitrile mixed with water or water containing buffer like, but not limited to, ammonium dihydrogen phosphate or an acid like phosphoric acid or trifluoracetic acid. The Solid Phase Extraction (SPE) is preferably performed on a Seppak® like, but not limited to, C18, tC18, Silica or an Oasis Seppak®. The compound is preferably eluted from the Seppak® with a solvent suitable for injection in vivo, like ethanol.

The above treated compounds may be formulated to a desired formulation for their intended use. For example the collected HPLC fraction from the preparative HPLC, containing a compound according to the invention may be diluted with water or water containing such as, but not limited to, sodium hydroxide or hydrogen chloride. The diluted fraction as prepared is trapped on a Seppak® like, but not limited to, C18, tC18, Silica or an Oasis Seppak® and the compound is preferably eluted from the Seppak® with a solvent suitable for injection in vivo, like ethanol. The obtained eluate is preferable diluted with pharmaceutically acceptable buffers such as, but not limited to 0.9% sodium chloride, sodiumdihydrogenphosphate 7.09 mM in 0.9% sodiumchloride or citrate buffer, pharmaceutically acceptable solubilisers such as, but not limited to, ethanol, tween or phospholipids and/or with pharmaceutically acceptable stabilizers or antioxidants such as, but not limited to, ascorbic acid, gentisic acid or p-aminobenzoic acid.

The invention shall be illustrated by means of the following non-limiting examples.

### EXAMPLE 1

Example 1 describes the preparation of 2-chloro-5-(methylthio)aniline hydrochloride (PK006) according to the below reaction equation:

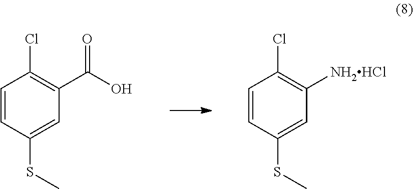

To a stirred solution of 2-chloro-5-(methylthio)benzoic acid (10.05 g, 49.6 mmol) in t-Butanol (40 mL) was added triethylamine (11 ml, 79 mmol). Diphenyl phosphorazidate (12 ml, 55.7 mmol) was added dropwise at a rate of one drop per sec. The reaction mixture was slowly heated and refluxed for 6 hours. The reaction mixture was cooled and the solvents were evaporated. The residue was dissolved in THF (25 mL) and hydrochloric acid/water (1:1) (25 mL) was added. The reaction mixture was refluxed for 6 hours and cooled to room temperature. The solvents were evaporated and the residue was taken up in ethyl acetate, the pH was adjusted with NaOH (25%) to 12. The mixture was extracted with ethyl acetate (4×50 ml). The combined organic fractions were combined and washed with water (30 ml). The organic layer was collected and dried with magnesium sulfate, filtered and evaporated to dryness. The residue was purified by column chromatography (EtoAc/PE 1:7). The fraction containing the product was evaporated and dissolved in ether, 2M hydrochloric acid in diethylether (20 mL) was added to the stirred solution. The HCl salt was collected by filtration. ¹H NMR (DMSO-d₆) δ 7.11 (d, 1H, H_{Aryl}, J=8.33 Hz), 6.72 (d, 1H, H_{Aryl}, J=2.27 Hz), 6.46 (dd, 1H, H_{Aryl}, J=8.32 Hz, J=2.25 Hz), 2.40 (s, 3H, Me).

### EXAMPLE 2

Example 2 describes in general the N-cyanation (a) according to the below equation:

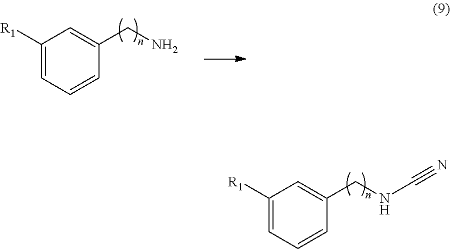

To a solution of the appropriately substituted aniline (1 equiv.) in ether (10 mL) at 0° C. was added dropwise a solution of cyanic bromide (2 equiv.) in ether (10 mL). After complete addition the mixture was warmed to ambient temperature and stirred 1-20 hours and followed by TLC. The solids were filtrated and washed with ether. The filtrate was washed with 1M HCl (25 mL) followed by brine (25 mL). The organic layer was collected, dried over anhydrous MgSO₄, filtrated and evaporated to dryness under reduced pressure.

### EXAMPLE 3

According to the general procedure of Example 2N-(3-hydroxyphenyl)cyanamide (PK112) was prepared from (91.63 mmol, 9.999 g) of the corresponding 3-aminophenol:

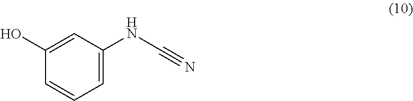

The N-(3-hydroxyphenyl)cyanamide (PK112) was obtained as a white solid (5.936 g, 44.25 mmol, 48%); R_{f} 0.39 (EtOAc:PE, 33:67). ¹H NMR (DMSO-d₆) [symbol] 9.98 (s, 1H, OH), 9.60 (bs, 1H, NH), 7.14-7.07 (m, 1H, H_{Aryl}), 6.44-6.36 (m, 3H, H_{Aryl}).

### EXAMPLE 4

According to the general procedure of Example 2N-(3-(difluoromethoxy)phenyl)cyanamide (PK070) was prepared from 3-(difluoromethoxy)aniline (1596 mg, 10.03 mmol).

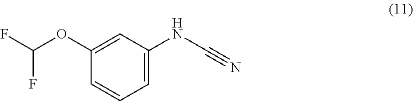 [SMILES: N#CNc1cccc(OC(F)F)c1]

After purification over silica with EtOAc/PE (25:75), N-(3-(difluoromethoxy)phenyl)cyanamide (PK070) was obtained as a light brown oil which solidifies on standing (683 mg, 3.71 mmol, 37%); R_{f} 0.24 (EtOAc:PE, 25:75). ¹H NMR (CDCl₃) δ 7.31-7.22 (m, 2H, H_{Aryl}), 6.87-6.76 (m, 2H, H_{Aryl}), 6.77 (s, 1H, NH), 6.48 (t, 1H, CHF₂, J=73.5 Hz).

### EXAMPLE 5

According to the general procedure of Example 2N-(3-(trifluoromethoxy)phenyl)cyanamide (PK069) was prepared from 3-(trifluoromethoxy)aniline (1780 mg, 10.05 mmol).

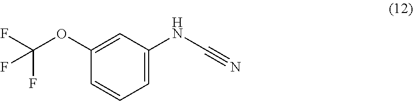 [SMILES: N#CNc1cccc(OC(F)(F)F)c1]

After purification over silica with EtOAc/PE (25:75) N-(3-(trifluoromethoxy)phenyl)cyanamide (PK069) was obtained as a white solid (583 mg, 2.88 mmol, 29%); R_{f} 0.43 (EtOAc:PE, 25:75). ¹H NMR (CDCl₃) δ 7.39-7.32 (m, 2H, H_{Aryl}), 6.99-6.93 (m, 2H, H_{Aryl}), 6.88 (s, 1H, NH).

### EXAMPLE 6

According to the general procedure of Example 2N-(3-(1,1,2,2-tetrafluoroethoxy)phenyl)cyanamide (PK212) was prepared from 3-(1,1,2,2-tetrafluoroethoxy)aniline (1.03 g, 4.94 mmol).

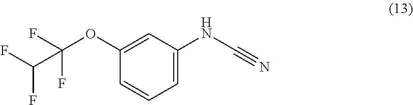 [SMILES: N#CNc1cccc(OC(F)(F)C(F)F)c1]

After purification over silica with EtOAc/PE (20/80) N-(3-(1,1,2,2-tetrafluoroethoxy)phenyl)cyanamide (PK212) was obtained as a colorless oil (475 mg, 2.03 mmol, 41%); R_{f} 0.22 (EtOAc/PE (60-80), 20/80, v/v). ¹H NMR (CDCl₃) δ 7.34 (t, 1H, H_{Aryl}, J=8.17 Hz), 6.98-6.89 (m, 3H, H_{Aryl},), 5.90 (dt, 1H, CHF₂, J=52.96, 2.85 Hz).

### EXAMPLE 7

Example 7 described in general the procedure for N-Methylation (B) according to the below equation:

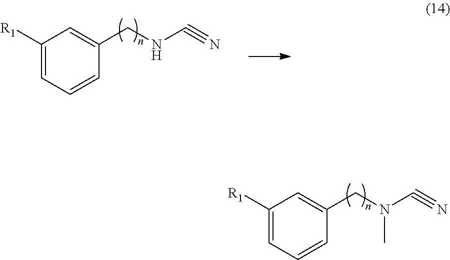

To a stirred solution at ambient temperature of the appropriately substituted cyanamine (1 equiv) in DMF (5 mL), as for example obtained by the general procedure of Example 2 or as in Examples 3-5 potassium carbonate (1.1 equiv.) was added. After 5 minutes methyl iodide was added (2 equiv.) and the mixture was stirred for 18 hours. The solvent was evaporated and the residue was dissolved in water (25 mL). The mixture was extracted with ethyl acetate (3×20 mL) and the combined organic fraction was dried over anhydrous MgSO₄, filtrated and evaporated to dryness under reduced pressure.

### EXAMPLE 8

According to the general procedure of Example 7N-(3-hydroxyphenyl)-N-methylcyanamide (PK113) was prepared starting from N-(3-hydroxyphenyl)cyanamide (PK112) (6.21 g, 43.76 mmol).

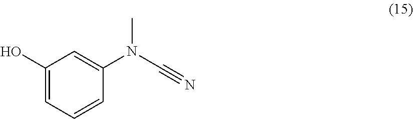

N-(3-hydroxyphenyl)-N-methylcyanamide was obtained as a yellow oil (4.21 g, 28.38 mmol, 65%); R_{f} 0.42 (EtOAc:PE, 33:67). ¹H NMR (CDCl₃) δ 9.71 (bs, 1H, OH), 7.23-7.16 (m, 1H, H_{Aryl}), 6.56-6.49 (m, 3H, H_{Aryl}), 3.27 (s, 3H, Me).

### EXAMPLE 9

According to the general procedure of Example 7N-(3-(difluoromethoxy)phenyl)-N-methylcyanamide (PK072) was prepared from N-(3-(difluoromethoxy)phenyl)cyanamide (PK070) (683 mg, 3.71 mmol)

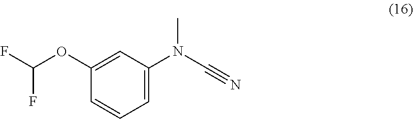

After purification over silica with EtOAc/PE (14:86), N-(3-(difluoromethoxy)phenyl)-N-methylcyanamide (PK072) was obtained as a yellow oil (477 mg, 2.41 mmol, 65%); R_{f} 0.48 (EtOAc:PE, 20:80). ¹H NMR (CDCl₃) δ 7.40-7.34 (t, J=8.18 Hz, 1H, H_{Aryl}), 6.98-6.94 (m, 1H, H_{Aryl}), 6.88-6.83 (m, 2H, H_{Aryl}), 6.77 (s, 1H, NH), 6.54 (t, ²J=73.5 Hz, 1H, CHF₂), 3.34 (s, 3H, NMe).

### EXAMPLE 10

According to the general procedure of Example 7N-methyl-N-(3-(trifluoromethoxy)phenyl)cyanamide (PK071) was prepared from N-(3-(trifluoromethoxy)phenyl)cyanamide (PK069) (583 mg, 2.88 mmol).

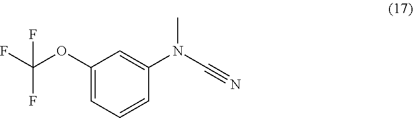

After purification over silica with EtOAc/PE (20:80), N-methyl-N-(3-(trifluoromethoxy)phenyl)cyanamide (PK071) was obtained as a colorless oil (384 mg, 1.78 mmol, 62%); R_{f} 0.49 (EtOAc:PE, 20:80). ¹H NMR (CDCl₃) δ 7.56 (t, 1H, H_{Aryl}, J=8.3 Hz), 7.22-7.07 (m, 3H, H_{Aryl}), 3.51 (s, 3H, CH₃).

### EXAMPLE 11

According to the general procedure of Example 7N-methyl-N-(3-(1,1,2,2-tetrafluoroethoxy)phenyl)cyanamide (PK214) was prepared from N-(3-(1,1,2,2-tetrafluoroethoxy)phenyl)cyanamide (PK212) (475 mg, 2.03 mmol).

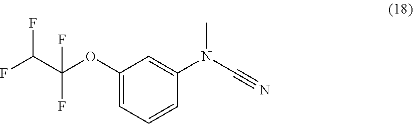 [SMILES: CN(C#N)c1cccc(OC(F)(F)C(F)F)c1]

After purification over silica with EtOAc/PE (20:80), N-methyl-N-(3-(1,1,2,2-tetrafluoroethoxy)phenyl)cyanamide (PK214) was obtained as a yellow oil (454 mg, 1.83 mmol, 90%); R_{f} 0.32 (EtOAc/PE (20:80). ¹H NMR (CDCl₃) δ 7.38 (t, 1H, H_{Aryl}, J=8.26 Hz), 7.04-6.89 (m, 3H, H_{Aryl}), 5.90 (dt, 1H, CHF₂, J=53.05, 2.82 Hz), 3.34 (s, 3H, CH₃).

### EXAMPLE 12

Example 12 described in general the procedure for the synthesis of the di- or tri-N-substituted guanidines according to the below equation:

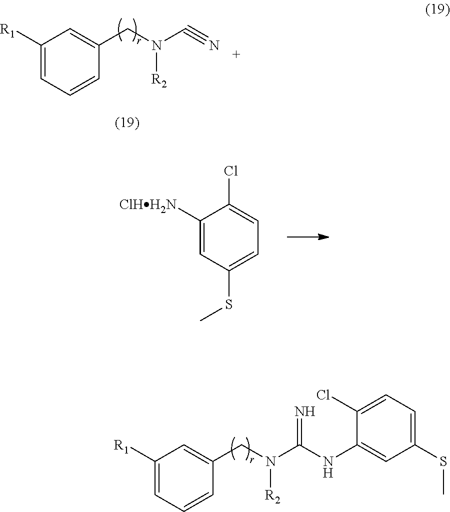

In a screw cap reaction vessel were dissolved the appropriately substituted cyanamide (1 mmol) as obtained in the examples above and an amine halogen salt, as obtained in example 1 (1.1 mmol, 1.1 equiv) in chlorobenzene (200 μL). The reaction vessel was flushed with N₂, closed and stirred at 165° C. for 4-18 hours. The reaction mixture was cooled down and dissolved in ethyl acetate (25 mL) and washed with 0.1M HCl (2×25 mL) followed by water (25 mL). The pH of the combined aqueous layers were adjusted with potassium carbonate to pH 10 and extracted with ethyl acetate (2×25 mL). The organic layers were collected and dried over anhydrous MgSO₄, filtrated and evaporated to dryness under reduced pressure. The crude compound was purified by column chromatography over silica gel. Oils were converted into the corresponding fumaric or hydrochloric salt.

### EXAMPLE 13

According to the general procedure of Example 12 3-(2-chloro-5-(methylthio)phenyl)-1-(3-(difluoromethoxy)phenyl)-1-methylguanidine (PK083) was prepared by reacting N-(3-(difluoromethoxy)phenyl)-N-methylcyanamide (PK072) (207 mg, 1.04 mmol) with 2-chloro-5-(methylthio)aniline hydrochloride (PK006) (242 mg, 1.15 mmol).

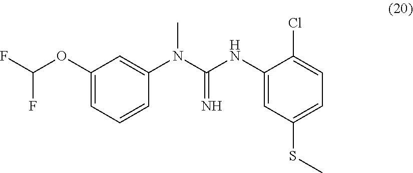 [SMILES: CSc1ccc(Cl)c(NC(=N)N(C)c2cccc(OC(F)F)c2)c1]

After purification over silica with EtOAc/PE/Et₃N (33:66:1), 3-(2-chloro-5-(methylthio)phenyl)-1-(3-(difluoromethoxy)phenyl)-1-methylguanidine (PK083) was obtained as a light yellow oil (182 mg, 0.49 mmol, 47%). R_{f} 0.09-0.33 (EtOAc:PE:Et₃N, 33:66:1). ¹H NMR (CDCl₃) δ 7.42-6.81 (m, 7H, H_{Aryl}), 6.56 (t, 1H, CHF₂, J=73.49 Hz), 3.96 (bs, 2H, NH), 3.42 (s, 3H, NCH₃), 2.46 (s, 3H, SCH₃). The free base was converted into it's fumaric acid salt (174 mg, 0.36 mmol, 80%). ¹H NMR (DMSO-d₆) δ 7.52-7.13 (m, 4H, H_{Aryl}), 7.22 (t, 1H, CHF₂, J=74.04 Hz), 6.96-6.93 (m, 1H, H_{Aryl}), 6.86-6.79 (m, 2H, H_{Aryl}), 6.59 (s, 2H, fumaric acid), 5.98 (bs, 2H, NH), 3.30 (s, 3H, NCH₃), 2.43 (s, 3H, SCH₃).

### EXAMPLE 14

According to the general procedure of Example 12 3-(2-chloro-5-(methylthio)phenyl)-1-methyl-1-(3-(trifluoromethoxy)phenyl)guanidine (PK082) was prepared by reaction of N-methyl-N-(3-(trifluoromethoxy)phenyl)cyanamide (PK071) (222 mg, 1.03 mmol) with 2-chloro-5-(methylthio)aniline hydrochloride (PK006) (236 mg, 1.12 mmol).

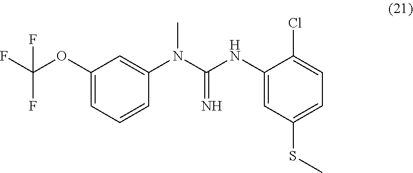 [SMILES: CSc1ccc(Cl)c(NC(=N)N(C)c2cccc(OC(F)(F)F)c2)c1]

After purification over silica with EtOAc/PE/Et₃N (16:83:1), 3-(2-chloro-5-(methylthio)phenyl)-1-methyl-1-(3-(trifluoromethoxy)phenyl)guanidine (PK082) was obtained as a white solid (213 mg, 0.55 mmol, 53%). R_{f} 0.16-0.28 (EtOAc:PE:Et₃N, 25:75:1). ¹H NMR (CDCl₃) δ 7.47-7.37 (m, 1H, H_{Aryl}), 7.32-7.23 (m, 3H, H_{Aryl}), 7.14-7.10 (m, 1H, H_{Aryl}), 6.93-6.83 (m, 2H, H_{Aryl}), 3.94 (bs, 2H, NH), 3.44 (s, 3H, NCH₃), 2.48 (s, 3H, SCH₃).

### EXAMPLE 15

According to the general procedure of Example 12 3-(2-chloro-5-(methylthio)phenyl)-1-(3-hydroxyphenyl)-1-methylguanidine (PK121) was prepared by reaction of N-(3-hydroxyphenyl)-N-methylcyanamide (PK113) (355 mg, 0.99 mmol) with 2-chloro-5-(methylthio)aniline hydrochloride (PK006) (232 mg, 1.10 mmol).

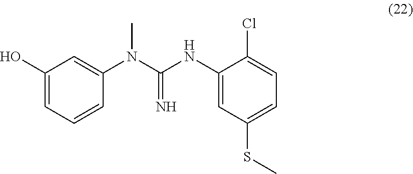 [SMILES: CSc1ccc(Cl)c(NC(=N)N(C)c2cccc(O)c2)c1]

After purification over silica with EtOAc/PE/Et₃N (50:50:1), 3-(2-chloro-5-(methylthio)phenyl)-1-(3-hydroxyphenyl)-1-methylguanidine (PK121) was obtained as a white solid (600 mg, 1.86 mmol, 78%). R_{f} 0.03-0.30 (EtOAc:PE:Et₃N, 50:50:1). ¹H NMR (CDCl₃) δ 7.51 (s, 1H, OH), 7.31-7.20 (m, 2H, H_{Aryl}), 6.95 (d, 1H, H_{Aryl}), 6.88-6.78 (m, 4H, H_{Aryl}), 5.21 (bs, 2H, NH), 3.42 (s, 3H, NCH₃), 2.48 (s, 3H, SCH₃).

### EXAMPLE 16

Example 16 describes the general procedure for the alkylation of the hydroxyguanidines according to the following equation:

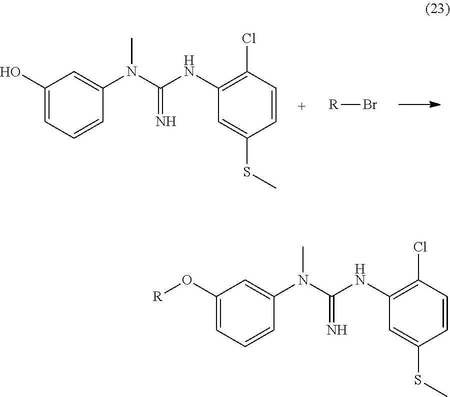

To a mixture of 3-(2-chloro-5-(methylthio)phenyl)-1-(3-hydroxyphenyl)-1-methylguanidine (PK121) (1 mmol, 1 equiv.) as obtained in Example 15, potassium carbonate (2 mmol, 2 equiv.) and potassium iodide (0.1 mmol, 0.1 equiv.) in DMF (2 mL) was added the appropriately alkylbromide (1-3 mmol, 1-3 equiv.). The reaction mixture was heated to 75° C. After 6 to 24 hours the reaction mixture was cooled to room temperature and diluted with water (25 mL) and washed twice with ethylacetate (25 mL). The combined organic layer was washed with brine (10 mL). The organic fraction was collected and dried with magnesiumsulfate, filtered and evaporated to dryness. The crude compound was purified by column chromatography over silica gel. Oils were converted into the corresponding fumaric or hydrochloric salt.

### EXAMPLE 17

According to the general procedure of Example 16 3-(2-chloro-5-(methylthio)phenyl)-1-(3-(3-fluoropropoxy)phenyl)-1-methylguanidine (PK134) was prepared by reaction of 3-(2-chloro-5-(methylthio)phenyl)-1-(3-hydroxyphenyl)-1-methylguanidine (PK121) (323 mg, 1.04 mmol) as obtained in Example 15 with 1-fluoro-3-bromopropane (0.1 mL, 1.09 mmol).

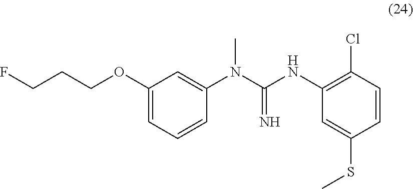 [SMILES: CSc1ccc(Cl)c(NC(=N)N(C)c2cccc(OCCCF)c2)c1]

After purification over silica with DCM/MeOH (95:5), 3-(2-chloro-5-(methylthio)phenyl)-1-(3-(3-fluoropropoxy)phenyl)-1-methylguanidine (PK134) was obtained as a brown oil (278 mg, 0.73 mmol, 73%). R_{f} 0.22 (DCM:MeOH, 95:5). ¹H NMR (CDCl₃) δ 7.33-7.25 (m, 2H, H_{Aryl}), 6.93-6.79 (m, 5H, H_{Aryl}), 4.66 (dt, 2H, FCH₂CH₂CH₂O, J=47.06 Hz, 5.75 Hz), 4.26 (bs, 2H, NH), 4.11 (t, 2H, FCH₂CH₂CH₂O, J=6.10 Hz), 3.43 (s, 3H, NCH₃), 2.46 (s, 3H, SCH₃), 2.19 (dq, 2H, FCH₂CH₂CH₂O, J=26.07 Hz, 5.91 Hz).The free base was converted into it's fumaric acid salt (168 mg, 0.37 mmol, 85%). ¹H NMR (DMSO-d₆) δ 7.31-7.24 (m, 2H, H_{Aryl}), 6.91-6.77 (m, 5H, H_{Aryl}), 6.58 (s, 1.81H, fumaric acid), 6.01 (bs, 2H, NH), 4.60 (dt, 2H FCH₂CH₂CH₂O, J=47.25 Hz, J=5.88 Hz), 4.07 (t, 2H, FCH₂CH₂CH₂O, J=6.18 Hz), 3.29 (s, 3H, NCH₃), 2.43 (s, 3H, SCH₃), 2.10 (dq, 2H, FCH₂CH₂CH₂O, J=25.80 Hz, 6.11 Hz).

### EXAMPLE 18

According to the general procedure of Example 12 1-(2-chloro-5-(methylthio)phenyl)-3-(3-(difluoromethoxy)phenyl)guanidine (PK176) was prepared by reacting N-(3-(difluoromethoxy)phenyl)cyanamide (PK070) (184 mg, 1.00 mmol) with 2-chloro-5-(methylthio)aniline hydrochloride (PK006) (231 mg, 1.10 mmol).

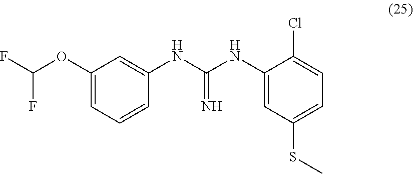 [SMILES: CSc1ccc(Cl)c(NC(=N)Nc2cccc(OC(F)F)c2)c1]

After purification over silica with DCM/MeOH (95:5), 1-(2-chloro-5-(methylthio)phenyl)-3-(3-(difluoromethoxy)phenyl)guanidine (PK176) was obtained as white crystals (200 mg, 0.56 mmol, 56%). R_{f} 0.33 (DCM/MeOH 95:5). ¹H NMR (CDCl₃) δ 7.29-7.20 (m, 2H, Ar), 7.11 (d, 1H, Ar, J=2.27 Hz), 7.02-7.00 (m, 1H, Ar), 6.86 (dd, 1H, Ar, J=8.41, 2.29 Hz), 6.45 (t, 1H, CHF₂, J=74.08 Hz), 5.42 (bs, 3H, NH), 2.43 (s, 3H, CH₃).

### EXAMPLE 19

According to the general procedure of Example 12 1-(2-chloro-5-(methylthio)phenyl)-3-(3-(trifluoromethoxy)phenyl)guanidine (PK191) was prepared by reacting N-(3-(trifluoromethoxy)phenyl)-N-cyanamide (PK069) (202 mg, 1.00 mmol) with 2-chloro-5-(methylthio)aniline hydrochloride (PK006) (231 mg, 1.10 mmol).

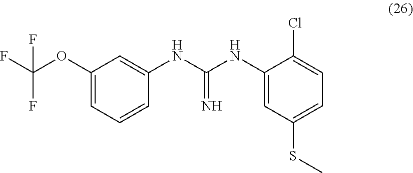 [SMILES: CSc1ccc(Cl)c(NC(=N)Nc2cccc(OC(F)(F)F)c2)c1]

After purification over silica with DCM/MeOH (95:5), 1-(2-chloro-5-(methylthio)phenyl)-3-(3-(difluoromethoxy)phenyl)guanidine (PK176) was obtained as white crystals (93 mg, 0.25 mmol, 25%). R_{f} 0.29 (DCM/MeOH 95:5). ¹H NMR (CDCl₃) δ 7.40 (d, 1H, H_{Aryl}, J=8.46 Hz), 7.28 (dt, 1H, H_{Aryl}, J=7.91, 0.58 Hz), 7.26 (d, 1H, H_{Aryl}, J=2.56 Hz), 7.15 (dd, 1H, H_{Aryl}, 8.46, 2.34 Hz), 6.93-6.81 (m, 3H, H_{Aryl}), 3.80 (bs, 2H, NH), 3.30 (s, 3H, NCH₃), 2.51 (s, 3H, SCH₃).

### EXAMPLE 20

According to the general procedure of Example 12 3-(2-chloro-5-(methylthio)phenyl)-1-methyl-1-(3-(1,1,2,2-tetrafluoroethoxy)phenyl)guanidine (PK217) was prepared by reacting N-methyl-N-(3-(1,1,2,2-tetrafluoroethoxy)phenyl)cyanamide (PK214) (248 mg, 1.00 mmol) with 2-chloro-5-(methylthio)aniline hydrochloride (PK006) (231 mg, 1.10 mmol).

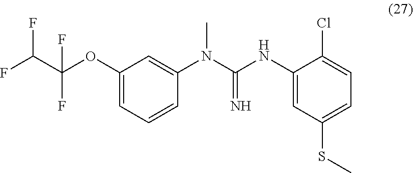 [SMILES: CSc1ccc(Cl)c(NC(=N)N(C)c2cccc(OC(F)(F)C(F)F)c2)c1]

after purification over silica with EtOAc/PE (50/503-(2-chloro-5-(methylthio)phenyl)-1-methyl-1-(3-(1,1,2,2-tetrafluoroethoxy)phenyl)guanidine was obtained as a light yellow oil (193 mg, 0.46 mmol, 46%). R_{f} 0.17 (EtOAc/PE 50/50). ¹H NMR (CDCl₃) δ 7.42 (t, 1H, H_{Aryl}, J=8.08 Hz), 7.30-7.21 (m, 3H, H_{Aryl}), 7.13-7.10 (m, 1H, H_{Aryl}), 6.91 (d, 1H, H_{Aryl}, J=2.23 Hz), 6.84 (dd, 1H, H_{Aryl}, J=8.34 Hz, 2.28 Hz), 5.94 (tt, 1H, CHF₂, J=53.05 Hz, 2.78 Hz), 3.92 (bs, 2H, NH), 3.43 (s, 3H, NCH₃), 2.47 (s, 3H, SCH₃). The free base was converted into its fumaric acid salt (135 mg, 0.22 mmol, 47%). ¹H NMR (DMSO-d₆) δ 7.44 (t, 1H, H_{Aryl}, J=8.14 Hz), 7.32-7.25 (m, 3H, H_{Aryl}), 7.06-6.99 (m, 2H, H_{Aryl}), 6.89-6.81 (m, 1H, H_{Aryl}), 6.79 (tt, 1H, CHF₂, J=51.93 Hz, 3.14 Hz), 6.61 s, 2H, fumaric acid), 6.17 (bs, 2H, NH), 3.32 (s, 3H, NCH₃), 2.44 (s, 3H, SCH₃)

### EXAMPLE 21

Example 21 described in general the procedure for the synthesis of [¹¹C]CH₃I starting from [¹¹C]CO₂. [¹¹C]CO₂ was trapped into a solution of LAIN in THF (0.1 mL) at room temperature by a helium flow of 10 mL·min⁻¹. The solution was heated to 130° C. and the helium flow was increased to 100 mL·min⁻¹ to evaporate the THF. After 3 min the helium flow was adjusted to 10 mL·min⁻¹, HI (55% solution, 0.2 mL) was added and [¹¹C]CH₃I was transferred into the reaction vial containing precursor, base and solvent.

### EXAMPLE 22

1-(2-chloro-5-(methylthio)phenyl)-3-(3-(difluoromethoxy)phenyl)guanidine (PK176) (0.5 mg, 1.40 μmol) was reacted with [¹¹C]CH₃I (as prepared in Example 21) in dimethylformamide (250 μL) in the presence of aqueous sodium hydroxide (5M, 5 μL) for 3 minutes at 80° C.

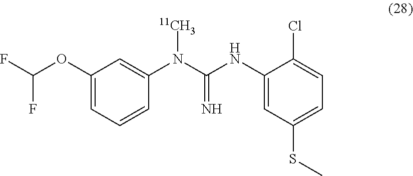 [SMILES: CSc1ccc(Cl)c(NC(=N)N([11CH3])c2cccc(OC(F)F)c2)c1]

After reaction the mixture is quenched with 10 mM NH₄OAc (pH=9.3, 300 μL) before purification by preparative HPLC. The fraction containing product was collected and diluted with water (50 mL). The solution was concentrated on a tC18plus Seppak, rinsed with water (20 mL), subsequently eluted with ethanol (96%, 1.5 mL) and diluted with a solution of 7.11 mM NaH₂PO₄ in 0.9% NaCl (w/v in water), pH 5.2 (13.5 mL) to give a final solution of 10% ethanol.

### EXAMPLE 23

1-(2-chloro-5-(methylthio)phenyl)-3-(3-(trifluoromethoxy)phenyl)guanidine (PK191) (0.5 mg, 1.40 μmol) was reacted with [¹¹C]CH₃I (as prepared in Example 21) in dimethylformamide (250 μL) in the presence of aqueous sodium hydroxide (5M, 5 μL) for 3 minutes at 80° C.

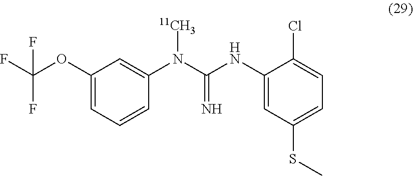 [SMILES: CSc1ccc(Cl)c(NC(=N)N([11CH3])c2cccc(OC(F)(F)F)c2)c1]

After reaction the mixture is quenched with 10 mM NH₄OAc (pH=9.3, 300 μL) before purification by preparative HPLC. The fraction containing product was collected and diluted with water (50 mL). The solution was concentrated on a tC18plus Seppak, rinsed with water (20 mL), subsequently eluted with ethanol (96%, 1.5 mL) and diluted with a solution of 7.11 mM NaH₂PO₄ in 0.9% NaCl (w/v in water), pH 5.2 (13.5 mL).

### EXAMPLE 24

Example 24 described in general the procedure for the synthesis of [¹⁸F]CH₂FBr. [¹⁸F]F⁻ was dried in the presence of K222 (15 mg) and potassium carbonate (2 mg).

A solution of CH₂Br₂ in MeCN (50%, 0.5 mL) was added and reacted for 5 minutes at 100° C. After reaction the synthesised [¹⁸F]CH₂FBr was distilled out of the reaction vessel by a helium flow of 50 mL·min⁻¹ via four coupled Seppak® silica Plus cartridges to purify the [¹⁸F] CH₂FBr and collected into a reaction vessel.

### EXAMPLE 25

Example 25 described in general the procedure for the synthesis of [¹⁸F]CH₂OTf. [¹⁸F]CH₂FBr was converted online to [¹⁸F]CH₂OTf by passing it trough a heated AgOTf column at 200° C. and collected into a reaction vessel.

### EXAMPLE 26

3-(2-chloro-5-(methylthio)phenyl)-1-(3-hydroxyphenyl)-1-methylguanidine (PK121) was reacted with either [¹⁸F]CH₂FBr (as prepared in Example 24) or with [¹⁸F]CH₂OTf (as prepared in Example 25) in dimethylformamide (250 μL) in the presence or absence of potassium iodide (0.5 mg) and sodium hydride (1 mg) for 15 minutes at 100° C.

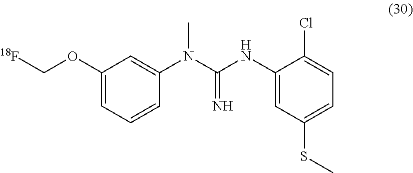 [SMILES: CSc1ccc(Cl)c(NC(=N)N(C)c2cccc(OC[18F])c2)c1]

After reaction the mixture is quenched with 25 mM NH₄H₂PO₄ (pH=2.5, 800 μL) before purification by preparative HPLC. The fraction containing product was collected and diluted with water (50 mL). The solution was concentrated on a tC18plus Seppak, rinsed with water (20 mL), subsequently eluted with ethanol (96%, 1 mL) and diluted with a solution of 7.11 mM NaH₂PO₄ in 0.9% NaCl (w/v in water), pH 5.2 (9 mL).

### EXAMPLE 27

### Membrane Preparation

Male Wistar rats (150-200 g) were killed by decapitation. The forebrains were rapidly removed and homogenized using a DUALL tissue homogenizer (10 strokes, 2000 rpm), in a 7-fold excess (v/w) of ice-cold 0.25 M sucrose. The nuclei and cell debris were removed by centrifugation (10 min x 400×g) in a Sorvall RC-6 refrigerated centrifuge (rotor SA600). The supernatant was decanted and the resulting pellet was rehomogenized in 5 vol 0.25 M sucrose and recentrifuged. The combined supernatants were diluted in Tris-acetate buffer (50 mM, pH 7.4) to a final dilution of 40 v/w, and centrifuged for 30 min×30,000×g, in order to obtain membranes from the cell surface, mitochondrial, and microsomal fractions. The pellet was resuspended in 20 vol of 50 mM Tris buffer containing 0.04% Triton X-100 (pH 7.4), and was kept at 25° C. for 2 hr before recentrifugation. The resulting pellet was suspended in Tris-HCl buffer (dilution 4, pH 7.4) and stored at −80° C. in 5 ml aliquots. On the day of each experiment, membranes were thawed to room temperature and washed twice by centrifugation (30 min×200,000×g). After the final centrifugation step, pellets were suspended in 50 mM Tris-HCl buffer (pH 7.4) and further diluted to 40 vol of buffer per g original weight wet tissue for competition binding experiments.

Protein concentration was determined using the BCA protein kit (Sigma-Aldrich, The Netherlands).

### EXAMPLE 28

### Competition Binding Assays

In vitro competition binding experiments were performed using 5 nM [³H]MK-801 (specific activity 22.5 Ci/mmol; PerkinElmer, USA). All compounds as prepared in the Examples and listed in Table 2 were dissolved as 10 mM stock solutions in DMSO, and used in a concentration range from 10⁻⁴ to 10⁻¹² M, with a maximal DMSO concentration of 1%. Competition binding experiments were conducted at room temperature, in a final volume of 500 μl assay buffer (50 mM Tris-HCl, pH 7.4), containing 1 μM L-glutamate and glycine. The incubation mixture was composed of 400 μl membrane suspension (protein concentration 1 μg/μL), 50 μL [³H]MK-801, 45 μl assay buffer and 5 μl unlabeled drug solution. Nonspecific binding was determined in the presence of 30 μM GMOM. Incubations were terminated overnight by filtration, using a 48-well Brandel harvester and Whatman GF/B filters, presoaked in 0.3% polyethyleneimine. The filters were washed three times with 3 ml of ice-cold Tris-HCl buffer (pH 7.4), and radioactivity was subsequently determined by liquid scintillation spectrometry in 5 mL of Optiphase-HiSafe 3, at an efficiency of 40%.

### EXAMPLE 29

### Data Analysis

Kᵢ values were determined by nonlinear regression analysis using the equation: log EC50=log [10̂log Ki*(1+RadioligandNM/HotKdNM)], (GraphPad Software Inc., San Diego, Calif.).

Table 1 below shows the affinity of compounds for the NMDAR ion channel against [³H]MK801. In this table the Kᵢ for the different compounds is stated. The Kᵢ as measured against 5 nM [³H]MK-801 and represent the mean±SEM Kᵢ values of 2-6 independent determinations, each conducted in triplicate, The indexes (b), (c) and (d) describe the form in which the compound was tested: (b) fumaric acid salt, (c) free base, (d) hydrochloric acid salt.

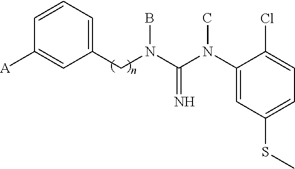

| A | n | B | C | no. | Kᵢ (nM)a |
|---|---|---|---|---|---|
| H | 0 | H | H | 1(b) | 722 ± 96 |
| H | 1 | H | H | 2(c) | >10 μM |
| H | 2 | H | H | 3(c) | >10 mM |
| OCH₃ | 0 | H | H | 4(b) | 136 ± 1.7 |
| OCH₃ | 1 | H | H | 5(d) | >1 μM |
| OCH₃ | 2 | H | H | 6(c) | >10 μM |
| OCHF₂ | 0 | H | H | 7(c) | 147 ± 15 |
| OCF₃ | 0 | H | H | 8(c) | 235 ± 55.5 |
| OCHF₂ | 0 | H | CH3 | 9(b) | 322 ± 19.5 |
| OCF₃ | 0 | H | CH3 | 10(c) | 650 ± 101 |
| H | 0 | CH3 | H | 11(b) | 308 ± 75 |
| H | 1 | CH3 | H | 12(c) | >10 μM |
| H | 2 | CH3 | H | 13(c) | >10 mM |
| OCH₃ | 0 | CH3 | H | 14(b) | 21.7 ± 2.2 |
| OCH₃ | 1 | CH3 | H | 15(d) | >1 μM |
| OCH₃ | 2 | CH3 | H | 16(c) | >10 μM |
| OH | 0 | CH3 | H | 17(c) | 551 ± 57.8 |
| OCHF₂ | 0 | CH3 | H | 18(b) | 10 ± 2 |
| OCF₃ | 0 | CH3 | H | 19(c) | 12 ± 2 |
| OCF₂CHF₂ | 0 | CH3 | H | 20(b) | 56.7 ± 6.2 |
| OCH₂F | 0 | CH3 | H | 21(b) | 18 ± 3 |
| O(CH₂)₂F | 0 | CH3 | H | 22(b) | 155 ± 34 |
| O(CH₂)₃F | 0 | CH3 | H | 23(b) | 179 ± 39 |

### EXAMPLE 30

### Biodistribution Studies

Male, 7-9 weeks old, B6C3 mice were used in all experiments. Animals were anaesthetized with an i.p. injection of Hypnorm/dormicum (12 ml/kg), after which [¹⁸F]PK209 (as prepared in Example 26) (19.0±1.1 MBq at t₀) or [¹¹C]PK083 (as prepared in Example 22) (24.4±3.5 MBq at t₀) was administered via the tail vein, in a saline solution containing 10% ethanol (5 ml/kg). Following the injections, mice were killed by cervical dislocation at 5, 10, 30, or 60 min (n=4−6). At each time point, blood was obtained by heart punctures and selected organs, including the heart, liver, kidneys, lungs and brain were removed. The brain was further dissected into prefrontal cortex, striatum, cerebral cortex, hippocampus and cerebellum. All organs and brain areas were weighed, and recovered radioactivity was determined with a Compugamma (LKB Wallac), using 5×10 μl aliquots of the injected formulation as standard. Results are expressed as the differential absorption ratio (DAR): (cpm recovered/g tissue)/(cpm injected/g body weight). Two-way repeated measures ANOVA, followed by LSD post-hoc analysis was used for between-region (or organ) comparisons of radiotracer uptake at different time points.

### [¹⁸F]PK209 Biodistribution Results

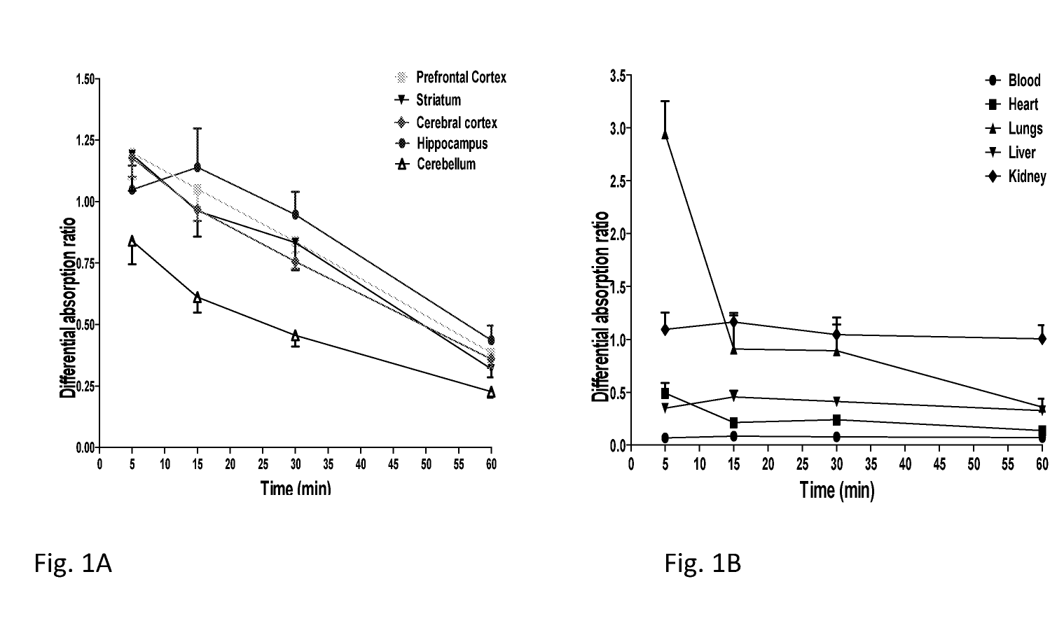

The brain uptake of [¹⁸F]PK209 was overall higher at 5 min, compared to all other time points (FIG. 1A; p<0.01, LSD posttests). DAR values at 5 min were 1.05±0.09, 1.17±0.07, 1.19±0.15, 1.19±0.10 and 0.84±0.09, for the hippocampus, cerebral cortex, striatum, prefrontal cortex and cerebellum, respectively. [¹⁸F]PK209 uptake was overall lower in the cerebellum, compared to all other brain areas analysed (p<0.01, LSD posttests). The highest ratio of radioactivity uptake between forebrain regions and the cerebellum was observed at 15 min post-injection. Radioactivity cleared rapidly from the brain, and no differences in regional distribution were observed 60 min following tracer injection. Two way ANOVA confirmed significant main effects of region [F_{(4,14)}=4.3, p<0.01] and time [F_{(3,42)}=49.9, p<0.001] on the brain uptake of [¹⁸F]PK209.

FIG. 1 shows the Biodistribution of [¹⁸F]PK209 in the CNS (FIG. 1A) and in selected organs (FIG. 1B). In FIG. 1A the boxes represent the differential absorption ratio results for the Prefrontal Cortex, the downwardly pointed triangles for the Striatum, the diamonds for the Cerebral cortex, the circles for the Hippocampus and the open upwardly pointed triangles for the Cerebellum. In FIG. 1B the circles represent the differential absorption ratio results for blood, the boxes for heart, the upwardly pointed triangles for lungs, the downwardly pointed triangles for liver and the diamonds for kidney.

The uptake of [¹⁸F]PK209 was higher in the lungs and the kidney, compared to all other organs (FIG. 1B; p<0.001, LSD posttests). The highest [¹⁸F]PK209 uptake was measured in the lungs, 5 min following tracer injection (p<0.001; LSD posttests), whereas the lowest levels of radioactivity were overall observed in the blood. The organ uptake of [¹⁸F]PK209 was higher at 5 min post-injection, compared to all other time points (p<0.001; LSD posttests). Activity was rapidly cleared from the lungs, and only the kidneys showed significantly higher levels of [¹⁸F]PK209 60 min post-injection, compared to other organs (p<0.01; LSD posttests). Two way ANOVA confirmed significant main effects of organ [F_{(4,15)}=51.1, p<0.001] and time [F_{(3,45)}=17.1, p<0.001], as well as significant organ x time interaction effects [F_{(12,45)}=12.1, p<0.001] on uptake of [¹⁸F]PK209.

### [¹¹C]PK083 Biodistribution Results

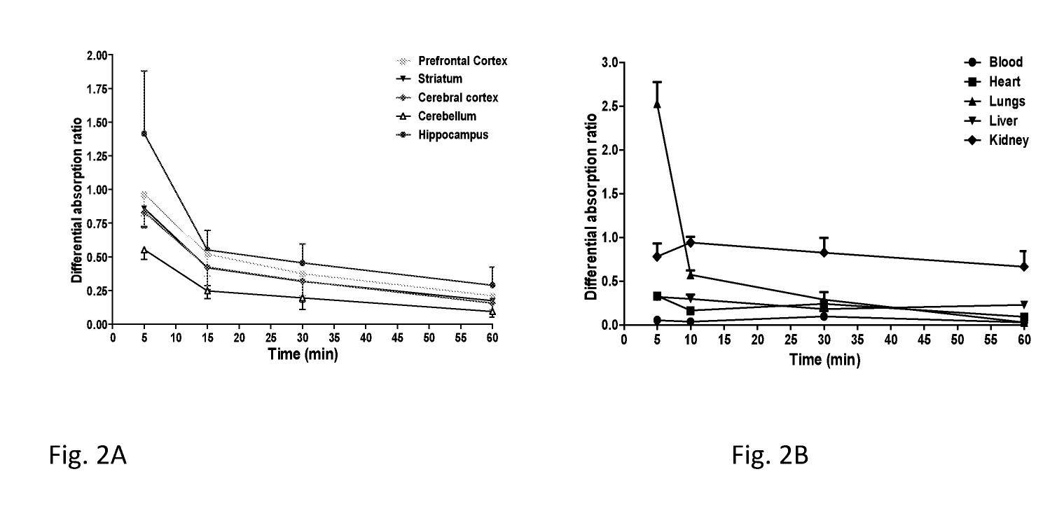

The brain uptake of [¹¹C]PK083 was higher at 5 min, compared to all other time points (FIG. 2A; p<0.001, LSD posttests). DAR values at 5 min were 1.41±0.46, 0.83±0.11, 0.86±0.13, 0.96±0.16 and 0.55±0.07, for the hippocampus, cerebral cortex, striatum, prefrontal cortex and cerebellum, respectively. The highest ratio of radioactivity uptake between forebrain regions and the cerebellum was observed at 5 min post-injection, whereas higher [¹¹C]PK083 uptake was observed in the hippocampus, compared to all other brain areas analysed (p<0.01, LSD posttests). Radioactivity was rapidly cleared from the brain, and no differences in regional distribution were observed 15 min following tracer injection. Two way ANOVA confirmed significant main effects of region [F_{(4,15)}=4.8, p<0.01] and time [F_{(3,45)=}11.6, p<0.001] on the brain uptake of [¹¹C]PK083.

FIG. 2 shows the Biodistribution of [¹¹C]PK083 in the CNS (FIG. 2A) and in selected organs (FIG. 2B). In FIG. 2A the boxes represent the differential absorption ratio results for the Prefrontal Cortex, the downwardly pointed triangles for the Striatum, the diamonds for the Cerebral cortex, the circles for the Hippocampus and the open upwardly pointed triangles for the Cerebellum. In FIG. 2B the circles represent the differential absorption ratio results for blood, the boxes for heart, the upwardly pointed triangles for lungs, the downwardly pointed triangles for liver and the diamonds for kidney.

The uptake of [¹¹C]PK083 was higher in the lungs and the kidney, compared to all other organs (FIG. 2B; p<0.001, LSD posttests). The highest [¹¹C]PK083 uptake was measured in the lungs, 5 min following tracer injection (p<0.001; LSD posttests), whereas the lowest levels of radioactivity were overall observed in the blood. The organ uptake of [¹¹C]PK083 was higher at 5 min post-injection, compared to all other time points (p<0.001; LSD posttests). Significantly higher levels of [¹¹C]PK083 uptake were observed in the kidney 60 min post-injection, compared to all other organs (p<0.01; LSD posttests). Two way ANOVA confirmed significant main effects of organ [F_{(4,12)}=130.1, p<0.001] and time [F_{(3,36)}=24.7, p<0.001] on uptake of [¹¹C]PK083, as well as significant organ x time interaction effects [F_{(12,365)}=8.3, p<0.001].

### EXAMPLE 31

### Ex Vivo Autoradiography & Blocking Study

To visualize the distribution and specificity of [¹⁸F]PK209 or [¹¹C]PK083 for NMDA receptors, anaesthetized mice were injected with either saline or MK-801 (0.6 mg/kg, i.p.), 10 min before a tail-vein injection of each of the radiolabeled compounds (n=2). 5 min following injection of [¹¹C]PK083 or 15 min after [¹⁸F]PK209 injection, mice were killed by cervical dislocation, and their brains were removed, frozen in liquid nitrogen and processed for quantitative autoradiography. Briefly, 20 μm coronal brain sections were cut at 300 μm intervals, from rostral to caudal areas. Sections from each mouse were opposed to Kodak Biomax MR-1 film, either immediately or following 2×30 sec washes in ice-cold Tris-HCl buffer (pH 7.4) and a dip in ice-cold demi water. Films were developed after 24 hr, and relevant optical density (ROD) values were obtained using the MCID software. Sections from control and MK-801 treated mice were processed in parallel. All brain regions were identified by reference to the mouse atlas of Franklin and Paxinos (2001).

### Results

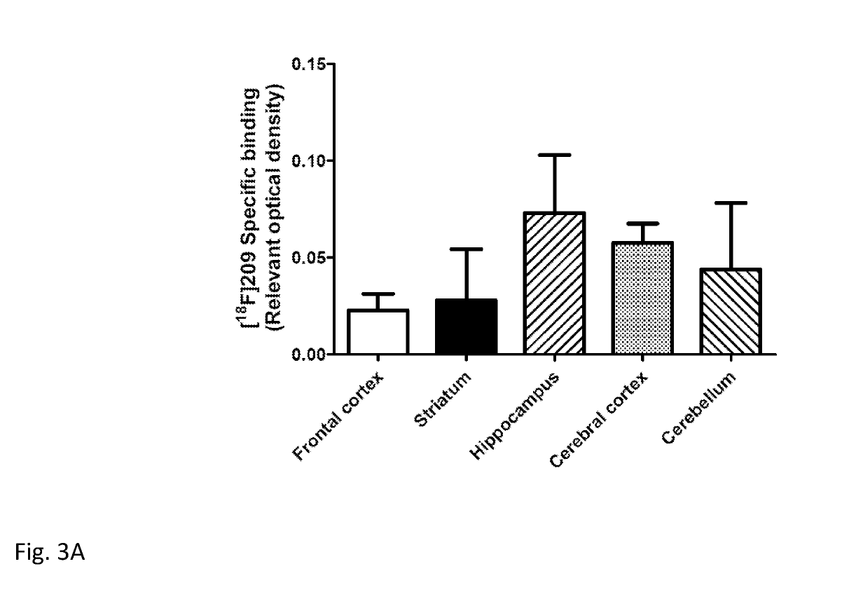

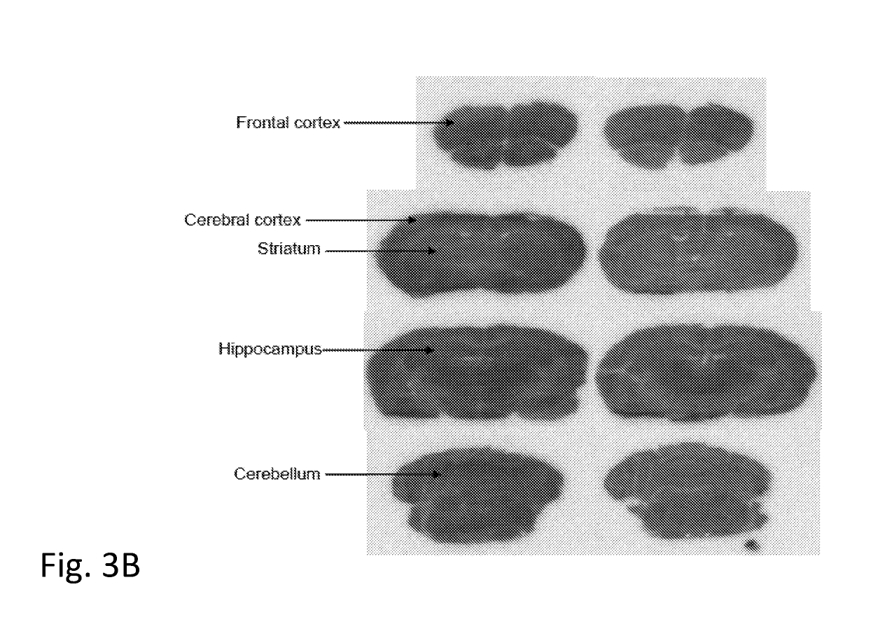

Reference is made to FIG. 3. FIG. 3 shows the quantitative autoradiography of [¹⁸F]PK-209 (FIG. 3A) and representative images (FIG. 3B). FIG. 3A the [¹⁸F]209 specific binding (relevant optical density) is shown for (from left to right) frontal cortex, striatum, hippocampus, cerebral cortex and cerebellum. In FIG. 3B representative images are shown for (from bottom to top) cerebellum, hippocampus, striatum, cerebral cortex and frontal cortex.

Specific binding was defined as that remaining following pretreatment with MK-801 (0.6 mg/kg i.p.). For [¹⁸F]PK-209, high binding levels were observed in the hippocampus and the cerebral cortex, followed by moderate levels in the cerebellum, and low levels in the frontal cortex and the striatum. Specific [¹⁸F]PK-209 binding constituted 10% and 8% of total binding values in the hippocampus and the cerebral cortex, respectively.

### EXAMPLE 32

### Determination of LogD_{oct,7.4}

The distribution of the radio labeled compounds between 1-octanol and 0.2M phosphate buffer (pH=7.4) was measured in triplicate at room temperature. Briefly, 1 mL of a 20 MBq/mL solution of the radio labeled compound in 0.2M phosphate buffer (pH=7.4) was vigorously mixed with 1 mL of 1-octanol for 1 min at room temperature using a vortex. After a settling period of 30 min, five samples of 100 μL were taken from both layers. For determining recovery, 5 samples of 100 μL were taken from the 20 MBq/mL solution. All samples were counted for radioactivity. The LogD_{oct,7.4} value was calculated according to LogD_{oct,7.4}=¹⁰Log(A_{oct}/A_{buffer}), where A_{oct} and A_{buffer} represent the average radioactivity counted of the 5 1-octanol and 5 buffer samples, respectively.

### RESULTS

[¹¹C]PK083: LogD_{oct,7.4} value=1.76±0.01

[¹⁸F]PK209: LogD_{oct,7.4} value=1.45±0.02

1. A N,N-substituted guanidine compound or a salt or solvate thereof according to formula (1) 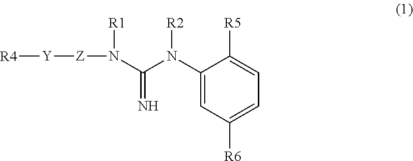
wherein R⁴ is a fluorinated organic group, Y is O, S or N,
Z is a substituted aryl group,
R¹ is methyl,
R² is hydrogen,
R⁵ is Cl or Br and
R⁶ is a thiomethyl group.

2. Compound according to claim 1, wherein the fluorinated organic group R⁴ has 1 to 5 carbon atoms.

3. Compound according to claim 2, wherein R⁴—Y— is a mono, bi or tri-fluorinated methoxy group.

4. Compound according to claim 1, wherein Z is a phenyl group substituted at its 3-position with the R⁴—Y— group.

5. Compound according to claim 1, wherein Y is O.

6. Compound according to claim 1, wherein R⁵ is Cl.

7. Compound according to claim 1, wherein R⁴—Y— is a mono, bi or tri-fluorinated methoxy group, Z is a phenyl group substituted at its 3-position with the R⁴—Y— group and R⁵ is Cl.

8. Compound according to claim 7, wherein R⁴—Y is a bi-fluorinated methoxy group or a tri-fluorinated methoxy group.

9. Compound according to claim 1, wherein at least one of groups R¹, R², R⁴, R⁵, R⁶ or the guanidine group contains a radio-isotope selected from ³H, ¹¹C, ¹⁸F, ⁷⁶Br, ¹²³I, ¹²⁴I, ¹²⁵I or ¹³¹I.

10. Compound according to claim 9, wherein one fluor atom in group R⁴ is the radio-isotope ¹⁸F or wherein the carbon of methyl group R¹ is the radio-isotope ¹¹C.

11. Compound according to claim 1 for use as an ion channel blocker of the N-methyl-D-aspartate (NMDA) receptor.

12. A radiopharmaceutical formulation comprising the compound according to claim 9.

13. A radiopharmaceutical formulation comprising the compound according to claim 9 for use as an in vivo diagnostic or imaging method.

14. A method for the in vivo diagnosis or imaging of NMDA related disease in a subject, preferably a human, comprising administration of a compound according to claim 9 or a formulation according to claim 12.

15. Process for the preparation of a N,N-substituted guanidine compound according to claim 10 by
(i) reaction of a precursor in a suitable solvent and in the presence of a base with an [¹⁸F]fluoroalkyl-L, wherein L is a leaving group; wherein the precursor is a compound according to formula (I) wherein the hydrogen group R² is optionally exchanged by an amine protecting group and wherein on the R4-Y position a hydroxyl group is present on the aryl group Z, and
(ii) removal of the protection amine group in a suitable solvent and protective group removing reagent provided that R² is exchanged by an amine protecting group.

16. Process according to claim 15, wherein the precursor is 3-(2-chloro-5-(methylthio)phenyl)-1-(3-hydroxyphenyl)-1-methylguanidine.

17. Process according to claim 15, wherein the leaving group L is a chlorine, bromine, iodine or a sulphonate ester leaving group.

18. A method comprising use of 3-(2-chloro-5-(methylthio)phenyl)-1-(3-hydroxyphenyl)-1-methylguanidine to prepare a ¹⁸F labelled compound according to claim 10.

19. A compound selected from 1-(2-chloro-5-(methylthio)phenyl)-3-(3-(difluoromethoxy)phenyl)guanidine or 1-(2-chloro-5-(methylthio)phenyl)-3-(3-(trifluoromethoxy)phenyl)guanidine or 3-(2-chloro-5-(methylthio)phenyl)-1-(3-hydroxyphenyl)-1-methylguanidine.

20. A method comprising use of 1-(2-chloro-5-(methylthio)phenyl)-3-(3-(difluoromethoxy)phenyl)guanidine or 1-(2-chloro-5-(methylthio)phenyl)-3-(3-(trifluoromethoxy)phenyl)guanidine to prepare a ¹¹C labeled 1-(2-chloro-5-(methylthio)phenyl)-3-¹¹C-methyl-3-(3-(difluoromethoxy)phenyl)guanidine or 1-(2-chloro-5-(methylthio)phenyl)-3-¹¹C-methyl-3-(3-(trifluoromethoxy)phenyl)guanidine respectively.
